(19) United States
(12) Patent Application Publication
(10) Pub. No.: US 2013/0345542 A1
(43) Pub. Date: Dec. 26, 2013

(54) DETERMINATION BY INDEPENDENT COMPONENT ANALYSIS OF THE EFFICACY OF A TREATMENT

(71) Applicants: Sylvain Mériadec Prigent, Biot (FR); Xavier Descombes, Antibes (FR); Josiane Zerubia, Cannes la Bocca (FR); Didier Zugaj, Mougins le Haut (FR)

(72) Inventors: Sylvain Mériadec Prigent, Biot (FR); Xavier Descombes, Antibes (FR); Josiane Zerubia, Cannes la Bocca (FR); Didier Zugaj, Mougins le Haut (FR)

(73) Assignees: Inria Institut National de Recherche en Informatique et en Automatique, Le Chesnay Cedex (FR); Galderma Research & Development, Biot (FR)

(21) Appl. No.: 13/980,857

(22) PCT Filed: Jan. 20, 2012
(86) PCT No.: PCT/EP12/50864

§ 371 Date: Sep. 11, 2013

(30) Foreign Application Priority Data
Jan. 20, 2011 (FR) 1150459

(51) Int. Cl. A61B 5/00

(52) U.S. Cl. CPC A61B 5/4848; A61B 5/0075; A61B 5/0077; A61B 2576/00
USPC 600/407

(57) ABSTRACT

A method is described for determining a value for quantifying the deviation from a mean value of a distribution of a divergence in severity between an initial instant and a later instant subsequent to the initial instant.

The technical field of the invention is statistical classification systems, and more particularly systems for statistical classification of hyperspectral images.

During clinical trial phases, the progression of skin diseases is quantified by dermatologists over an entire period of treatment. In a first phase, the degree to which a patient is affected by the disease is measured on each patient of a group. The measurement is carried out clinically by a dermatologist. In a second phase, a statistical treatment of the measurements makes it possible to quantify the efficacy of the treatment.

In practice, an operating protocol based on the study over time of the symptoms associated with a skin disease, which are expressed in a group of Ne patients, is used. Each patient receives a treatment on a first zone of affected skin and a vehicle on a second zone of affected skin. The first zone of skin and the second zone of skin are chosen so as to have a similar surface area and to be similarly affected by the disease. In the case of a disease affecting the face, one cheek receives the treatment, while the other cheek receives the vehicle, under the condition that the two cheeks are equally affected by a skin disease.

A dermatologist thus considers the degree to which a patient is affected by the disease, zone by zone, patient by patient. As a result, the quantification of the efficacy of the treatment may be empirical and subject to a certain degree of subjectivity.

In order to improve the observation and the quantification of the degree to which a patient is affected by a disease while at the same time increasing the reproducibility of these steps, hyperspectral imaging can be used. It is recalled that hyperspectral imaging consists in acquiring several images at different wavelengths.

This is because chemical materials and elements react more or less differently when exposed to radiation of a given wavelength. By scanning the range of radiations, it is possible to differentiate materials involved in the composition of an object according to their difference of interaction. This principle can be generalized to a landscape, or to a part of an object.

The set of images resulting from the photograph of one and the same scene at different wavelengths is referred to as a hyperspectral image or hyperspectral cube.

A hyperspectral image therefore consists of a set of images of which each pixel is characteristic of the intensity of the interaction of the scene observed at a particular wavelength. By knowing the interaction profiles of the materials with various radiations, it is possible to determine the materials present. The term “material” should be understood in a broad sense, covering not only solid, liquid and gaseous materials, but also pure chemical elements and complex assemblies of molecules or macromolecules.

The acquisition of hyperspectral images can be carried out according to several methods.

The method of acquiring hyperspectral images known as spectral scan consists in using a CCD sensor to produce spatial images, and in applying different filters in front of the sensor in order to select a wavelength for each image. Various filter technologies make it possible to meet the needs of such imagers. Mention may, for example, be made of liquid crystal filters which isolate a wavelength through electrical stimulation of the crystals, or acousto-optical filters which select a wavelength by deforming a prism due to a difference in electrical potential (piezo-electricity effect). These two filters have the advantage of not having mobile parts which are often a source of fragility in optical systems.

The method for acquiring hyperspectral images referred to as spatial scan aims to acquire or “to image” simultaneously all of the wavelengths of the spectrum on a CCD sensor. In order to implement the breakdown of the spectrum, a prism is placed in front of the sensor. A line-by-line spatial scan is then carried out in order to make up the complete hyperspectral cube.

The method for acquiring hyperspectral images referred to as spatial scan consists in carrying out an interference measurement, then in reconstituting the spectrum by carrying out a Fast Fourier Transform (FFT) on the interference measurement. The interference is implemented using a Michelson system, which causes a ray to interfere with itself with a temporal offset.

The final method for acquiring hyperspectral images aims to combine the spectral scan and the spatial scan. Thus, the CCD sensor is partitioned in the form of blocks. Each block therefore processes the same region of the space but with different wavelengths. A spectral and spatial scan then allows a complete hyperspectral image to be constituted.

When applied to dermatological studies, hyperspectral imaging enables the acquisition of images comprising information linked to the wavelength. The intensity of each pixel as a function of the wavelength is recorded. The application of classification methods to these images makes it possible to distinguish the healthy zones and affected zones. Mention may be made of the studies by P. Comon, “Independent component analysis: a new concept?,” Signal Processing, Elsevier, vol. 36, pp. 287-314, 1994 regarding a method of independent component analysis allowing signal classification.

The classification of hyperspectral images is a particularly active field. Several algorithms exist for processing and classifying hyperspectral images obtained on the skin.

I. L. Weatherall and B. D. Coombs, “Skin color measurements in terms of CIELAB color space value,” Journal of Investigative Dermatology, vol. 99, pp. 468-473, 1992 teach color image processing by CIEL*a*b breakdown.

G. N. Stamatas et al., “Non-invasive measurements of skin pigmentation in situ.” Pigment cell res, vol. 17, pp. 618-626, 2004 teach that the L* component or the ITA value calculated with the L* and b* components makes it possible to describe the pigmentation.

G. N. Stamatas et al., “In vivo measurement of skin erythema and pigmentation: new means of implementation of diffuse reflectance spectroscopy with a commercial instrument,” British Journal of Dermatology, vol. 159, pp. 683-690, 2008 describes the separation of the contributions of melanin and of hemoglobin in a hyperspectral image on the basis of the empirical study of their respective absorptions.

S. Prigent et al., “Spectral analysis and unsupervised SVM classification for skin hyper-pigmentation classification,” IEEE Workshop on Hyperspectral Image and Signal Processing: Evolution in Remote Sensing (Whispers), Reykjavik, Iceland, June 2010 and S. Prigent et al., “Multi-spectral image analysis for skin pigmentation classification,” Proc. IEEE International Conference on Image Processing (ICIP), Hong Kong, China, September 2010 describe methods of classifying healthy zones and diseased zones using hyperspectral images.

By acquiring other hyperspectral images at various instants, it is possible to add temporal information. It then becomes possible to observe the progression of a dermatological disease over time. Finally, by performing statistical analysis of the results of a panel of individuals, it is possible to determine the efficacy of a treatment on the disease observed, more particularly in pigmentary disorders, acne, rosacea or psoriasis. This determination can be extended to hyperspectral images of skin appendages, in particular of skin appendages suffering from mycoses, for instance onychomycosis. The term “skin appendages” is intended to mean the nails and the hair. Among the skin appendages, interest is directed more particularly to the nails.

At the current time, a notable effect is acknowledged only after a statistical study on a wide panel of patients. In order to process the data resulting from the various images at various instants for the various patients, it is necessary to have an effective image processing system.

An objective of the invention is to generate images having a maximum contrast between images of a zone affected by a disease and images of a zone spared by the disease.

Another objective of the invention is to determine a numerical index reflecting the efficacy of the treatment of a disease.

Another objective of the invention is an image processing system capable of determining a numerical index reflecting the efficacy of the treatment of a disease.

A subject of the invention is a method of determining a value for quantifying the deviation from the mean value of a distribution of the divergence in severity between an initial instant and a later instant subsequent to the initial instant, the severity depending on the contrast between zones receiving a treatment and zones receiving a vehicle. The method comprises steps in the course of which:

at least one hyperspectral image comprising at least one zone chosen from a diseased zone receiving the treatment, a healthy zone receiving the treatment, a diseased zone receiving the vehicle and a healthy zone receiving the vehicle is acquired, for at least one patient, at an initial instant and at at least one later instant subsequent to the initial instant,

a breakdown of the hyperspectral images into independent components is determined,

a representative component maximizing the divergence between healthy zone and pathological zone is determined, for each image, from among the independent components,

the linear combination of the spectral bands corresponding to the representative component is stored, for each image,

the mean over all the images, of the absolute values of the linear combinations of the spectral bands each corresponding to the representative component of an image is determined,

a representative component corrected as a function of the mean of the representative components is determined for each image, a value of the divergence in severity is determined as a function of the corrected representative components of the hyperspectral images acquired, for each patient and for each measurement instant, and

a value for quantifying the deviation from the mean value between a distribution of the divergence in severity at the initial instant and a distribution of the divergence in severity at a later instant subsequent to the initial instant is determined as a function of the value of the divergence in severity for each patient at the initial instant and at the later instant.

The invention has the advantage of providing a single numerical index for characterizing the efficacy of the treatment between two measurement instants automatically and using only the hyperspectral images of a set of patients, the hyperspectral images being classified between images of a healthy zone and images of a pathological zone.

It is possible to determine a value of the divergence in severity for a measurement instant,

by determining, for each of the images acquired, a mean intensity equal to the mean value of the pixels for the corrected representative component,

by determining a value of the severity of the disease for a patient at a measurement instant, calculated for the images relating to the zones having received the treatment concerning the patient and the measurement instant considered,

by determining a value of the severity of the disease for the patient considered at the measurement instant considered, calculated for the images relating to the zones having received the vehicle, concerning the patient considered and the measurement instant considered, and

by determining a value of the divergence in severity of the disease between the zones having received the treatment and the zones having received the vehicle for a patient at a measurement instant.

It is possible to determine the value of the severity of the disease for a patient at a measurement instant, calculated for the images relating to the zones having received the treatment, concerning the patient considered and the measurement instant considered, by simple differencing of the mean intensity value for the image of the healthy zone having received the treatment and of the mean intensity for the image of the diseased zone having received the treatment, and

it is possible to determine the value of the severity of the disease for the patient considered at the measurement instant considered, calculated for the images relating to the zones having received the vehicle, concerning the patient considered and the measurement instant considered, by simple differencing of the mean intensity for the image of the healthy zone having received the vehicle and of the mean intensity for the image of the diseased zone having received the vehicle.

It is possible to determine the value of the severity of the disease for the patient considered at the measurement instant considered, calculated for the images relating to the zones having received the treatment, concerning the patient considered and the measurement instant considered, by relative differencing of the mean intensity for the image of the healthy zone having received the treatment and of the mean intensity for the image of the diseased zone having received the treatment, and

it is possible to determine the value of the severity of the disease for the patient considered at the measurement instant considered, calculated for the images relating to the zones having received the vehicle, concerning the patient considered and the measurement instant considered, by relative differencing of the mean intensity for the image of the healthy zone having received the vehicle and of the mean intensity for the image of the diseased zone having received the vehicle.

It is possible to determine the value of the severity of the disease for the patient considered at the measurement instant considered, calculated for the images relating to the zones having received the treatment, concerning the patient considered and the measurement instant considered, as being equal to the mean intensity for the image of the diseased zone having received the treatment, and

it is possible to determine the value of the severity of the disease for the patient considered at the measurement instant considered, calculated for the images relating to the zones having received the vehicle, concerning the patient considered and the measurement instant considered, as being equal to the mean intensity for the image of the diseased zone having received the vehicle.

It is possible to determine a value of the divergence in severity of the disease between the zones having received the treatment and the zones having received the vehicle for the patient considered at the measurement instant considered by simple differencing of the severity of the disease for the patient considered at the measurement instant considered, calculated for the images relating to the zones having received the treatment, and the value of the severity of the disease for the patient considered at the measurement instant considered, calculated for the images relating to the zones having received the vehicle.

It is possible to determine a value of the divergence in severity of the disease between the zones having received the treatment and the zones having received the vehicle for the patient considered at the measurement instant considered by relative differencing of the severity of the disease for the patient considered at the measurement instant considered, calculated for the images relating to the zones having received the treatment, and the value of the severity of the disease for the patient considered at the measurement instant considered, calculated for the images relating to the zones having received the vehicle.

The vehicle may be a placebo.

The vehicle may be another treatment.

Another subject of the invention is an image processing system for determining the severity of a disease, comprising a hyperspectral image acquisition device connected to a processing means, the processing means being connected to a data storage means and to a man-machine interaction device, the processing means being capable of applying the method defined above.

Another subject of the invention is the application of the method described above to the determination of the efficacy of a dermatological treatment.

Other objectives, features and advantages will emerge on reading the following description given only by way of nonlimiting example and produced with reference to the appended figures in which:

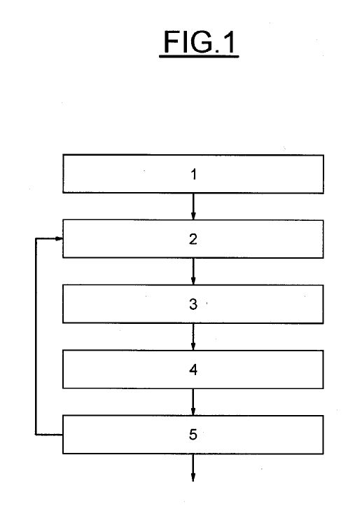

FIG. 1 illustrates the method of determination according to the invention, and

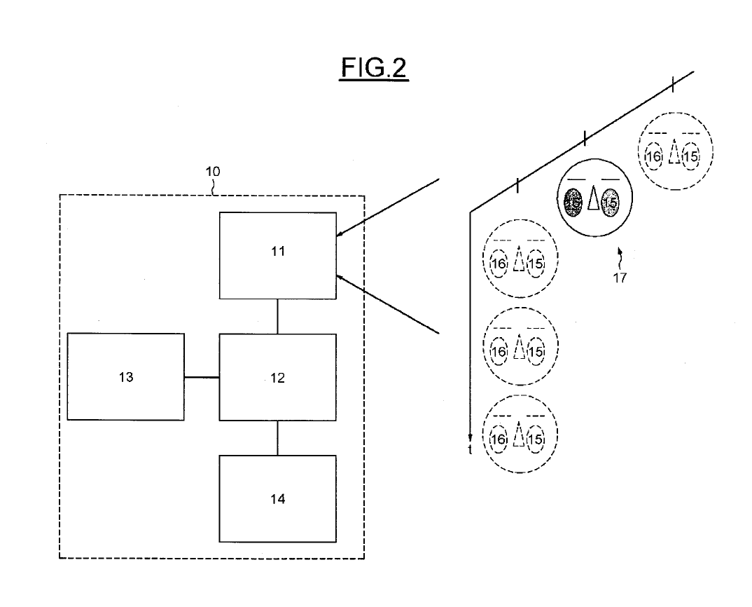

FIG. 2 illustrates the associated image processing system.

The method of determination begins with the acquisition 1, of at least one hyperspectral image comprising a zone from among a diseased zone receiving the treatment, a healthy zone receiving the treatment, a diseased zone receiving the vehicle and a healthy zone receiving the vehicle. These hyperspectral images are acquired at each measurement instant and for each patient. In the rest of the description, it will be considered that the acquisition of four images each comprising a zone of interest has been carried out.

However, it is possible to have at least two zones of interest distributed on one image. The image can then be divided up into as many subimages as there are zones of interest present on the initial image. In this case, care will be taken to obtain subimages having the same number of pixels. Care will also be taken to redimension the hyperspectral images which have not benefited from being cut up into subimages to an image size having the same number of pixels as the subimages.

Those skilled in the art will thus be capable of adapting the method described hereinafter to the case where at least one image comprises several zones of interest by inserting a step of creating one subimage per zone of interest and of redirnensioning the images which are not subjected to being cut up. In any event, the method is provided with four hyperspectral images each corresponding to a zone of interest.

The term “vehicle” is intended to mean a composition comprising the same excipients as those corresponding to the treatment, but not comprising the active ingredients which act on the causes of the disease. The vehicle may also be a placebo or a control solution.

The vehicle may be another treatment, i.e. a composition comprising active ingredients and excipients different than the composition of the first treatment.

Although the method described is based on the application of a treatment and of a vehicle, it is possible to apply the method in order to compare a zone receiving a first treatment with a zone receiving a second treatment.

The four hyperspectral images are processed by means of an independent component analysis (ICA) method. Each image is converted by the ICA method into an image of the same size and comprising as many independent components as the original image comprised wavelengths. Each of the independent components results from a linear combination of the wavelengths of the original image.

A representative component maximizing the divergence between healthy zone and pathological zone is determined, for each image, from among the independent components.

The linear combination of the spectral bands corresponding to the representative component is stored, for each image. The term “linear combination” is intended to mean the weighting coefficients of each spectral band. Variations in coefficients may exist from one patient to another. In order to obtain a reference, the mean of the absolute values of the weighting coefficients which are part of the linear combination of the representative component of each patient is calculated. The mean of the absolute values could also be calculated while discarding beforehand the aberrant or extreme values. Mean coefficients are thus obtained.

A representative component corrected as a function of the mean of the representative components, i.e. as a function of the mean coefficients, is determined for each image.

The spatial extension of the disease is not taken into account. Thus, the method of determination comprises a step 2 of determining a mean value of the pixels of the corrected representative component M, for each image.

At the end of this step, four mean values (μ_{Mh}^{V}, μ_{Mp}^{V}, μ_{Mh}^{A}, and μ_{Mp}^{A}) are obtained, each corresponding to one of the images received during the preceding step. These values are determined at each measurement instant and for each patient.

The mean intensity on a corrected representative component M of a diseased zone receiving the treatment is denoted μ_{Mp}^{A}.

The mean intensity on a corrected representative component M of a healthy zone receiving the treatment is denoted μ_{Mh}^{A}.

The mean intensity on a corrected representative component M of a diseased zone receiving the vehicle is denoted μ_{Mp}^{V}.

The mean intensity on a corrected representative component M of a healthy zone receiving the vehicle is denoted μ_{Mh}^{V}.

A mean intensity denoted μ_{M} can be determined via the following equation:

$${μ}_{M} = {( Mean ( I ) )}_{M} = {\sum }_{m = 1}^{N p} I ( m ; M ) \frac{1}{N p} \qquad ( Eq . 1 )$$

With (Mean(I))_{M}=the mean intensity relating to the corrected representative component M

Nb: the total number of bands

Np: the total number of pixels per band of the image

I(m,M): the intensity of the pixel m of the corrected representative component M.

The method of determination then determines a single value for quantifying the disease of a patient using the four mean intensities determined in the preceding step. The single value obtained is the severity Dₜ^{e}. In order to obtain the severity Dₜ^{e}, a first normalization applied between the diseased zone and the healthy zone is carried out in the course of a step 3 of the method, followed by a second normalization in the course of another step 4 of the method.

The first normalization can be carried out by simple differencing.

dₜ^{e}=μ_{Mh}−μ_{Mp} (Eq. 2)

Alternatively, the first normalization can be carried out by relative differencing.

$${d}_{t}^{e} = \frac{{μ}_{Mh} - {μ}_{Mp}}{{μ}_{Mh}} \qquad ( Eq . 3 )$$

with

dₜ^{e}: the severity of the disease for the patient e at the measurement instant t,

μ_{Mh}: the mean intensity for a healthy zone,

μ_{Mp}: the mean intensity for a diseased zone.

According to another alternative, the first normalization can correspond to the mean measurement for the diseased zone

dₜ^{e}=μ_{Mp} (Eq. 4)

Since the measurements μ_{Mh} and μ_{Mp} are homogeneous, the normalization equation 2 is preferred. These values are determined at each measurement instant and for each patient.

At the end of this step, a severity of zones having received the treatment dₜ^{e,A} is obtained if the values μ_{Mh} and μ_{Mp} are replaced with the values μ_{Mh}^{A} and μ_{Mp}^{A} relating to the mean intensities for treated zones. Likewise, a severity of zones having received the vehicle dₜ^{e,V} is obtained if the values μ_{Mh} and μ_{Mp} are replaced with the values μ_{Mh}^{V} and μ_{Mp}^{V} relating to the mean intensities for zones having received the vehicle.

The method of determination applies equation 2 while replacing the values μ_{Mh} and μ_{Mp} with the values μ_{Mh}^{A} and μ_{Mp}^{A} in order to obtain the severity dₜ^{e,A}.

dₜ^{e,A}=(dₜ^{e})^{A}=(μ_{Mh}−μ_{Mp})^{A}=μ_{Mh}^{A}−μ_{Mp}^{A} (Eq. 5)

By analogy, the severity dₜ^{e,V} is obtained by replacing the values μ_{Mh} and μ_{Mp} of equation 5 with the values μ_{Mh}^{V} and μ_{Mp}^{V}.

Alternatively, the method of determination applies equation 3 while replacing the values μ_{Mh} and μ_{Mp} with the values μ_{Mh}^{A} and μ_{Mp}^{A} in order to obtain the severity dₜ^{e,A}.

$${d}_{t}^{e , A} = {( {d}_{t}^{e} )}^{A} = {( \frac{{μ}_{Mh} - {μ}_{Mp}}{{μ}_{Mh}} )}^{A} = \frac{{μ}_{Mh}^{A} - {μ}_{Mp}^{A}}{{μ}_{Mh}^{A}} \qquad ( Eq . 6 )$$

By analogy, the severity dₜ^{e,V} is obtained by replacing the values μ_{Mh}^{A} and μ_{Mp}^{A} of equation 6 with the values μ_{Mh}^{V} and μ_{Mp}^{V}.

Alternatively, the method of determination applies equation 4 while replacing the values μ_{Mh} and μ_{Mp} with the values μ_{Mh}^{A} and μ_{Mp}^{A} in order to obtain the severity dₜ^{e,A}.

dₜ^{e,A}=(dₜ^{e})^{A}=μ_{Mp}^{A} (Eq. 7)

By analogy, the severity dₜ^{e,V} is obtained by replacing the value μ_{Mp}^{A} of equation 7 with the value μ_{Mp}^{V}. With

μ_{Mh}^{V}: the mean intensity for a healthy zone having received the vehicle,

μ_{Mp}^{V}: the mean intensity for a diseased zone having received the vehicle,

μ_{Mh}^{A}: the mean intensity for a healthy zone having received the treatment,

μ_{Mp}^{A}: the mean intensity for a diseased zone having received the treatment.

For the reasons cited with regard to the equations Eq. 2, Eq. 3 and Eq. 4, the normalization preferred here is that relating to the equation Eq. 5.

In order to obtain the severity Dₜ^{e} from the two severities dₜ^{e,A} and dₜ^{e,V}, a second normalization is applied between the zone receiving the treatment and the zone receiving the vehicle.

The second normalization makes it possible to determine the severity Dₜ^{e} of the patient e at the measurement instant t resulting from the comparison of the treatment and of the vehicle. From the severities dₜ^{e,A} of zones having received the treatment and from the severities dₜ^{e,V} of zones having received the vehicle, it is possible to determine a severity Dₜ^{e} for the patient e at the measurement instant t.

Dₜ^{e}=dₜ^{e,A}−dₜ^{e,V} (Eq. 8)

Alternatively, it is possible to determine a severity Dₜ^{e} for the patient e at the measurement instant t according to the following equation:

$${D}_{t}^{e} = \frac{{d}_{t}^{e , A} - {d}_{t}^{e , V}}{{d}_{t}^{e , V}} \qquad ( Eq . 9 )$$

with

dₜ^{e,A}: the severity of the disease for the patient e at the measurement instant t, calculated for the images relating to the zones having received the treatment,

dₜ^{e,V}: the severity of the disease for the patient e at the measurement instant t, calculated for the images relating to the zones having received the vehicle.

The method of determination applies equation 8 in order to determine the severity Dₜ^{e} for the patient e at the measurement instant t. Alternatively, equation 9 is applied in order to determine the severity Dₜ^{e} for the patient e at the measurement instant t.

For the reasons cited with regard to the equations Eq. 2, Eq. 3 and Eq. 4, the normalization preferred here is that relating to the equation Eq. 8. These values are determined at each measurement instant and for each patient.

At this stage, the distribution of the severities Dₜ^{e} between the various patients is not yet taken into account. In order to take it into account, a statistical analysis is necessary. For this, the inventors have ingenuously applied a t-test method to the data characterizing the divergence between the diseased zone treated and the diseased zone receiving the vehicle. In other words, the t-test is applied to the severity index Dₜ^{e}.

The t-test is in particular described in the book by A. M. Mood, F. A. Graybill, and D. C. Boes, “Introduction to the theory of statistics”, McGraw-Hill, 1974. This book discloses a method of characterizing the deviation from the mean value between two distributions. The method is called the Student's test, or t-test.

$${Z}^{t , {t}_{0}} ( t ) = \frac{\overline{X} ( t ) - \overline{X} ( {t}_{0} )}{\sqrt{\frac{{σ}^{2} ( t )}{{N}_{e}} + \frac{{σ}^{2} ( {t}_{0} )}{{N}_{e}}}} \qquad ( Eq . 12 )$$

With Z^{t,t}^{₀}(t) the quantification of the deviation from the mean value between two distributions, X̅(t) the mean value of X, σ(t) the standard deviation of X, Nₑ the number of patients in the group, t the measurement instant and t₀ the reference measurement instant. The null hypothesis is that the mean value of the distribution does not change between the time t₀ and the time t. The null hypothesis is rejected if the quantification of the deviation Z^{t,t0}(t) between the time t₀ and the time t has a probability (obtained according to the Student's law) less than the value p=0.05.

Moreover, the lower the value of the quantification of the deviation, the greater the divergence between the mean values of the two distributions between the instant t and the instant t₀.

This test is therefore applied to the severities Dₜ^{e} obtained at the end of the optimization of the divergence between healthy zone and diseased zone, X̅(t) then being the mean value, over all of the patients considered, of the severities Dₜ^{e} at the instant t, and σ^{t}(t) then being the standard deviation of the distribution of the severities Dₜ^{e} at the instant t.

The mean value of the severities Dₜ^{e} can be calculated in several ways known to those skilled in the art, for example a simple mean, a relative mean or a statistical mean. Furthermore, it is possible to remove the extreme values, or the aberrant values. In the latter two cases, the standard deviation of the distribution of the severities Dₜ^{e} is calculated by discarding the same values removed from all the values considered for producing the mean.

Alternatively, a paired t-test defined in the manner described hereinafter is considered. Once the optimized value \hat{λ} has been determined, the value of the severity Dₜ^{e} corresponding to the optimized value \hat{λ} is again determined for each measurement instant and for each patient, and used for the subsequent steps of the method.

At this stage, the distribution of the severities Dₜ^{e} between the various patients is not yet taken into account. In order to take it into account, a statistical analysis is necessary. For this, the inventors have ingenuously applied a paired t-test method to the data characterizing the divergence between the diseased zone treated and the diseased zone receiving the vehicle. In other words, the paired t-test is applied to the severity index Dₜ^{e}.

The method is called the paired Student's test, or paired t-test.

$${Z}^{t , {t}_{0}} ( t ) = \frac{\overline{X} ( t )}{\frac{σ ( t )}{\sqrt{{N}_{e}}}} \qquad ( Eq . 13 )$$

With Z^{t,t}^{₀}(t) the quantification of the deviation from the mean value between the distribution X and a standardized normal distribution N(0.1), X̅(t) the mean value of X, σ(t) the standard deviation of X, Nₑ the number of patients in the group, t the measurement instant and t₀ the reference measurement instant. The null hypothesis is that the mean value of the distribution does not change between the time t₀ and the time t. The null hypothesis is rejected if the quantification of the deviation Z^{t,t0}(t) between the time t₀ and the time t has a probability (obtained according to the Student's law) less than the value p=0.05.

Moreover, the lower the value of the quantification of the deviation, the greater the divergence between X and N(0.1).

This test is therefore applied to the severities Dₜ^{e} obtained at the end of the optimization of the divergence between healthy zone and diseased zone, X̅(t) then being the mean value, over all of the patients considered, of the difference Dₜ^{e}−Dₜ_{₀}^{e} between the instant t and the instant t₀, and σ(t) then being the standard deviation of the distribution of the difference Dₜ^{e}−Dₜ_{₀}^{e} between the instant t and the instant t₀.

The value of the difference Dₜ^{e}−Dₜ_{₀}^{e} between the instant t and the instant t₀ can be calculated in several ways known to those skilled in the art, for instance a simple mean, a relative mean or a statistical mean. Furthermore, it is possible to remove the extreme values, or the aberrant values. In the latter two cases, the standard deviation of the distribution of the severities Dₜ^{e}−Dₜ_{₀}^{e} is calculated by discarding the same values removed from all the values considered for producing the mean.

The method of determination thus comprises a step 5 of determining a value Z^{t,t0}(t) for the quantification of the deviation from the mean value of the distribution of the severities Dₜ^{e} between the time t₀ and the time t. This quantification value will be, on the one hand, compared to the null hypothesis for determining the presence of an effect, and then compared to values Z^{t,t0}(t) of other treatments in order to compare the effects thereof, or compared to values Z^{t,t0}(t) at other instants in order to determine the change over time.

If the divergence between a value of the Student's statistic associated with Z^{t,t0}(t) and the value 0.05 of the null hypothesis is high, this means that the treated zone changes increasingly distinctly from the nontreated zone. The treatment is then considered to be effective. The Student's statistic is obtained by reading the Student's law table. For a value of Z along the x-axis, there is a corresponding probability along the y-axis.

The value Z makes it possible to characterize the efficacy of the treatment over the total treatment duration. The value Z also makes it possible to compare the efficacy of a treatment with the efficacy of another treatment of the same duration.

The hyperspectral image processing system 10 comprises a hyperspectral image acquisition device 11 connected to a processing means 12, itself connected to a data storage means 13 and to a man-machine interaction device 14.

The acquisition device is capable of producing hyperspectral images of zones (15, 16) of a patient 17. The zones of which the image is acquired are a healthy zone 15 and a diseased zone 16. The images are also taken on fractions of these zones having received a treatment or a vehicle. The acquisition is repeated for several subjects and at different measurement instants. The data obtained are sent to the processing means 12 which processes them in real time or redirects them to the data storage means 13 for delayed processing.

The processing means 12 applies the steps of the method of determining a value for quantifying the deviation from the mean value of a distribution of the divergence in severity between an initial instant and a later instant subsequent to the initial instant, the severity depending on the contrast between zones receiving a treatment and zones receiving a vehicle.

The results of the treatment are displayed by means of the man-machine interaction device 14. The result can be displayed on a screen, sent to another system so as to be the subject of another processing, or sent via a remote electronic communication means to or several users.

1. A method of determining a value for quantifying a deviation from a mean value of a distribution of a divergence in severity between an initial instant and a later instant subsequent to the initial instant, the severity depending on the contrast between zones receiving a treatment and zones receiving a vehicle, the method comprising the following steps:
acquiring, for at least one patient, at in initial instant and at least one later instant subsequent to the initial instant, at least one hyperspectral image comprising at least one zone selected from the group consisting of a diseased zone receiving treatment, a healthy zone receiving treatment, a diseased zone receiving vehicle and a healthy zone receiving vehicle,
determining a breakdown of the hyperspectral images into independent components,
determining a representative component maximizing the divergence between healthy zone and pathological zone, for each image, from among the independent components,
storing a linear combination of spectral bands corresponding to a representative component, for each image,
determining the mean over all the images, of absolute values of the linear combinations of the spectral bands each corresponding to the representative component of an image,
determining a representative component corrected as a function of the mean of the representative components for each image,
determining a value of the divergence in severity as a function of the corrected representative components of the hyperspectral images acquired, for each patient and for each measurement instant, and
determining a value for quantifying the deviation from the mean value between a distribution of the divergence in severity at the initial instant and a distribution of the divergence in severity at a later instant subsequent to the initial instant as a function of the value of the divergence in severity for each patient at the initial instant and at the later instant.

2. The method as claimed in claim 1, wherein the value for quantifying the deviation from the mean value between a distribution of the divergence in severity at the initial instant and a distribution of the divergence in severity at a later instant subsequent to the initial instant is determined by applying a Student's test or a paired Student's test to the optimized values of the value of the divergence in severity for each patient at the initial instant and at the later instant.

3. The method as claimed in claim 1, wherein the value of the divergence in severity is determined for a measurement instant
by determining, for each of the images acquired, a mean intensity equal to the mean value of the pixels for the corrected representative component,
by determining a value of the severity of the disease for a patient at a measurement instant, calculated for the images relating to the zones having received the treatment concerning the patient and the measurement instant considered,
by determining a value of the severity of the disease for the patient considered at the measurement instant considered, calculated for the images relating to the zones having received the vehicle, concerning the patient considered and the measurement instant considered, and
by determining a value of the divergence in severity of the disease between the zones having received the treatment and the zones having received the vehicle for a patient at a measurement instant.

4. The method as claimed in claim 1, wherein the value of the severity of the disease for a patient at a measurement instant, calculated for the images relating to the zones having received the treatment, concerning the patient considered and the measurement instant considered, is determined by simple differencing of the mean intensity for the image of the healthy zone having received the treatment and of the mean intensity for the image of the diseased zone having received the treatment, and
the value of the severity of the disease for the patient considered at the measurement instant considered, calculated for the images relating to the zones having received the vehicle, concerning the patient considered and the measurement instant considered, is determined by simple differencing of the mean intensity for the image of the healthy zone having received the vehicle and of the mean intensity for the image of the diseased zone having received the vehicle.

5. The method as claimed in claim 1, wherein the value of the severity of the disease for the patient considered at the measurement instant considered, calculated for the images relating to the zones having received the treatment, concerning the patient considered and the measurement instant considered, is determined by relative differencing of the mean intensity for the image of the healthy zone having received the treatment and of the mean intensity for the image of the diseased zone having received the treatment, and
the value of the severity of the disease for the patient considered at the measurement instant considered, calculated for the images relating to the zones having received the vehicle, concerning the patient considered and the measurement instant considered, is determined by relative differencing of the mean intensity for the image of the healthy zone having received the vehicle and of the mean intensity for the image of the diseased zone having received the vehicle.

6. The method as claimed in claim 1, wherein the value of the severity of the disease for the patient considered at the measurement instant considered, calculated for the images relating to the zones having received the treatment, concerning the patient considered and the measurement instant considered, is determined as being equal to the mean intensity for the image of the diseased zone having received the treatment, and
the value of the severity of the disease for the patient considered at the measurement instant considered, calculated for the images relating to the zones having received the vehicle, concerning the patient considered and the measurement instant considered, is determined as being equal to the mean intensity for the image of the diseased zone having received the vehicle.

7. The method as claimed in claim 4, wherein a value of the divergence in severity of the disease between the zones having received the treatment and the zones having received the vehicle for the patient considered at the measurement instant considered is determined by simple differencing of the severity of the disease for the patient considered at the measurement instant considered, calculated for the images relating to the zones having received the treatment, and the value of the severity of the disease for the patient considered at the measurement instant considered, calculated for the images relating to the zones having received the vehicle.

8. The method as claimed in claim 4, wherein a value of the divergence in severity of the disease between the zones having received the treatment and the zones having received the vehicle for the patient considered at the measurement instant considered is determined by relative differencing of the severity of the disease for the patient considered at the measurement instant considered, calculated for the images relating to the zones having received the treatment, and the value of the severity of the disease for the patient considered at the measurement instant considered, calculated for the images relating to the zones having received the vehicle.

9. The method as claimed in claim 1, wherein the vehicle is a placebo.

10. The method as claimed in claim 1, wherein the vehicle is another treatment.

11. An image processing system for determining the severity of a disease, the system comprising a hyperspectral image acquisition device (11) connected to a processing means (12), the processing means (12) being connected to a data storage means (13) and to a man-machine interaction device (14), the processing means (12) being capable of applying the method as claimed in claim 1.

12. A method of determining efficacy of a therapeutic treatment, the method comprising applying the method of claim 1 to evaluation of the therapeutic treatment compared to vehicle treatment.
